(19) United States
(12) Patent Application Publication
(10) Pub. No.: US 2008/0243240 A1
(43) Pub. Date: Oct. 2, 2008

(54) Biodegradable Metal Barrier Layer for a Drug-Eluting Stent

(75) Inventors: David Doty, Forestville, CA (US); Michael Krivoruchko, Forestville, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/691,253

(22) Filed: Mar. 26, 2007

(51) Int. Cl. A61F 2/82

(52) U.S. Cl. 623/1.42

(57) ABSTRACT

An implantable medical device includes a substrate, a drug-impregnated layer deposited over the substrate, and a barrier layer at least partially covering the drug-impregnated layer. The barrier layer may be a biodegradable metal, biodegradable metal oxide, or biodegradable metal alloy, such as, magnesium, a magnesium oxide or a magnesium alloy. The drug-impregnated layer includes a therapeutic substance, such as, antineoplastic, anti-inflammatory, antiplatelet, anticoagulant, fibrinolytic, thrombin inhibitor, antimitotic, antiallergic, and antiproliferative substances.

### FIELD OF THE INVENTION

The present invention relates to implantable medical devices that release a drug, in particular, stents that provide in situ controlled release delivery of a therapeutic substance.

### BACKGROUND OF THE INVENTION

Cardiovascular disease, specifically atherosclerosis, remains a leading cause of death in developed countries. Atherosclerosis is a multifactorial disease that results in a narrowing, or stenosis, of a vessel lumen. Briefly, pathologic inflammatory responses resulting from vascular endothelium injury causes monocytes and vascular smooth muscle cells (VSMCs) to migrate from the sub endothelium and into the arterial wall's intimal layer. There the VSMC proliferate and lay down an extracellular matrix causing vascular wall thickening and reduced vessel patency.

Cardiovascular disease caused by stenotic coronary arteries is commonly treated using either coronary artery by-pass graft (CABG) surgery or angioplasty. Angioplasty is a percutaneous procedure wherein a balloon catheter is inserted into the coronary artery and advanced until the vascular stenosis is reached. The balloon is then inflated, restoring arterial patency. A variation in the angioplasty procedure may include arterial stent deployment. Briefly, after arterial patency has been restored, the balloon is deflated and a vascular stent is inserted into the vessel lumen at the stenosis site. After expansion of the stent, the catheter is then removed from the coronary artery and the deployed stent remains implanted to prevent the newly opened artery from constricting spontaneously. An alternative procedure, which is sometimes referred to as primary stenting, involves stent deployment without prior balloon angioplasty, wherein the expansion of the stent against the arterial wall is sufficient to open the artery and restore arterial patency. However, balloon catheterization and/or stent deployment can result in vascular injury ultimately leading to VSMC proliferation and neointimal formation within the previously opened artery. This biological process whereby a previously opened artery becomes re-occluded is referred to as restenosis.

Treating restenosis requires additional, generally more invasive, procedures including CABG surgery in severe cases. Consequently, methods for preventing restenosis, or treating incipient forms, are being aggressively pursued. One possible method for preventing restenosis is the administration of anti-inflammatory compounds that block local invasion/activation of monocytes thus preventing the secretion of growth factors that may trigger VSMC proliferation and migration. Other potentially anti-restenotic compounds include antiproliferative agents, such as chemotherapeutics, which include rapamycin and paclitaxel. Other classes of drugs such as anti-thrombotics, anti-oxidants, platelet aggregation inhibitors and cytostatic agents have also been suggested for anti-restenotic use.

However, many of these drugs, particularly anti-inflammatory and antiproliferative compounds, can be toxic when administered systemically in anti-restenotic-effective amounts. Accordingly, local delivery is a preferred method of treatment since smaller amounts of medication are administered in comparison to systemic dosages and the medication may be concentrated at a specific treatment site. Local delivery thus produces fewer side effects and achieves more effective results.

A common technique for local delivery of drugs involves coating a stent or graft with a polymeric material which, in turn, is impregnated with a drug or a combination of drugs. Once the stent or graft is implanted within a lumen of the cardiovascular system, the drug(s) is released from the polymer for treatment of the local tissues. The drug(s) is released into the lumen by a process of diffusion through the polymer layer for biostable polymers, and/or as the polymer material degrades for biodegradable polymers.

In attempts to control the rate of elution of a drug from the drug impregnated polymeric material, barrier layers have been provided. Barrier layers have generally been another layer of polymeric material. By providing an extra layer of polymeric material, it is thought that the elution rate can be controlled because the barrier layer adds material and distance through which the drug must diffuse to be released. However, test data has shown that the use of a polymeric barrier layer does not significantly slow elution.

U.S. Pat. No. 6,716,444 discloses a stent including a drug-impregnated polymeric layer over a substrate material, and further including a metallic barrier layer or cap coat. However, the metallic barrier layer of U.S. Pat. No. 6,716,444 is not biodegradable. Using a non-biodegradable metallic barrier or cap layer with a biodegradable base polymer is not desirable because as the drug-impregnated polymer degrades, the non-biodegradable metallic barrier or cap layer may fracture or collapse. The fracture or deformation of the metallic cap layer may then cause tissue inflammation or other complications at the artery wall.

Further, stent design is evolving to where a substrate material may be a biodegradable polymer or biodegradable metallic material. Accordingly, it would be desirable to have a biodegradable drug-impregnated layer and a biodegradable metallic barrier or cap layer such that the entire structure is biodegradable.

### BRIEF SUMMARY OF THE INVENTION

The present invention allows for a controlled rate of release of a drug or drugs from a polymer carried on an implantable medical device. The controlled rate of release allows localized drug delivery for extended periods, depending upon the application. This is especially useful in providing therapy to reduce or prevent cell proliferation, inflammation, or thrombosis in a localized area.

An embodiment of an implantable medical device in accordance with the present invention includes a substrate, which may be, for example, a metal or polymeric stent or graft, among other possibilities. At least a portion of the substrate is coated with a first layer that includes one or more therapeutic substances in a polymer carrier. A barrier layer overlies the first layer. The barrier layer reduces the rate of release of the therapeutic substance from the polymer once the medical device has been placed into the patient's body, thereby allowing an extended period of localized drug delivery once the medical device is in situ.

The barrier layer may be a biodegradable metal, biodegradable metal oxide, or biodegradable metal alloy and may have a thickness ranging from about 5 to about 100 nanometers. In various embodiments, a material of the barrier layer may be magnesium, a magnesium oxide or a magnesium alloy.

The one or more drugs contained within the drug-impregnated polymer layer may include, but are not limited to, antineoplastic, anti-inflammatory, antiplatelet, anticoagulant, fibrinolytic, thrombin inhibitor, antimitotic, antiallergic, and antiproliferative substances.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is a perspective view of an exemplary stent in accordance with an embodiment of the present invention.

FIG. 2 illustrates a cross-sectional view taken along line A-A of FIG. 1 of a stent strut.

FIG. 3 illustrates a cross-sectional view taken along line A-A of FIG. 1 of a stent strut in accordance with another embodiment of the present invention.

FIG. 4 illustrates a cross-sectional view along line A-A of FIG. 1 of a stent strut in accordance with another embodiment of the present invention.

### DETAILED DESCRIPTION OF THE INVENTION

Specific embodiments of the present invention are now described with reference to the figures, where like reference numbers indicate identical or functionally similar elements.

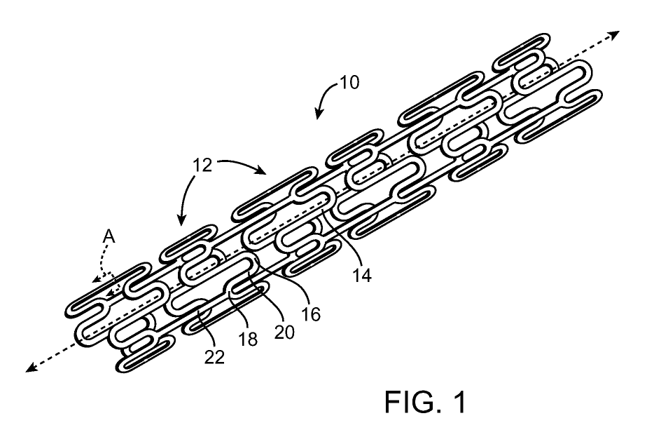

The present invention provides a stent or graft, which are often referred to as endoprostheses, with a drug-impregnated coating and a barrier or cap layer. FIG. 1 illustrates an exemplary stent 10 in accordance with an embodiment of the present invention. Stent 10 is a patterned tubular device that includes a plurality of radially expandable cylindrical rings 12. Cylindrical rings 12 are formed from struts 14 formed in a generally sinusoidal pattern including peaks 16, valleys 18, and generally straight segments 20 connecting peaks 16 and valleys 18. Connecting links 22 connect adjacent cylindrical rings 12 together. In FIG. 1, connecting links 22 are shown as generally straight links connecting a peak 16 of one ring 12 to a valley 18 of an adjacent ring 12. However, connecting links 22 may connect a peak 16 of one ring 12 to a peak 16 of an adjacent ring, or a valley 18 to a valley 18, or a straight segment 20 to a straight segment 20. Further, connecting links 22 may be curved. Connecting links 22 may also be excluded, with a peak 16 of one ring 12 being directly attached to a valley 18 of an adjacent ring 12, such as by welding, soldering, or the manner in which stent 10 is formed, such as by etching the pattern from a flat sheet or a tube. It will be appreciated by one of ordinary skill in the art that stent 10 of FIG. 1 is merely an exemplary stent and that stents of various forms and methods of fabrication can be used. For example, in a typical method of making a stent, a thin-walled, small diameter metallic tube is cut to produce the desired stent pattern, using methods such as laser cutting or chemical etching. The cut stent may then be descaled, polished, cleaned and rinsed. Some examples of methods of forming stents and structures for stents are shown in U.S. Pat. No. 4,733,665 to Palmaz, U.S. Pat. No. 4,800,882 to Gianturco, U.S. Pat. No. 4,886,062 to Wiktor, U.S. Pat. No. 5,133,732 to Wiktor, U.S. Pat. No. 5,292,331 to Boneau, U.S. Pat. No. 5,421,955 to Lau, U.S. Pat. No. 5,935,162 to Dang, U.S. Pat. No. 6,090,127 to Globerman, and U.S. Pat. No. 6,730,116 to Wolinsky et al., each of which is incorporated by reference herein in its entirety.

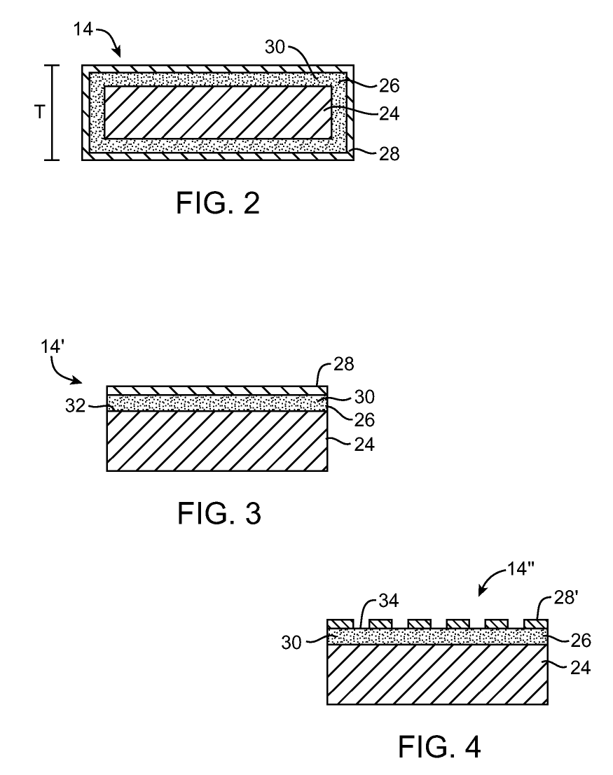

FIG. 2 is a cross-sectional view taken at A-A of FIG. 1 through a portion of strut 14 of stent 10. Strut 14 has a suitable thickness T that, typically, may be in the range of approximately 50 μm (0.002 inches) to 200 μm (0.008 inches). As shown in FIG. 2, strut 14 is formed of a substrate 24, a drug-impregnated layer 26, and a barrier layer 28. Substrate 24 may be any material that is typically used for a stent, for example, stainless steel, “MP35N,” “MP20N,” nickel titanium alloys such as Nitinol, tantalum, platinum-iridium alloy, gold, magnesium, L605, or combinations thereof. “MP35N” and “MP20N” are trade names for alloys of cobalt, nickel, chromium and molybdenum available from standard Press Steel Co., Jenkintown, Pa. “MP35N” consists of 35% cobalt, 35% nickel, 20% chromium, and 10% molybdenum. “MP20N” consists of 50% cobalt, 20% nickel, 20% chromium, and 10% molybdenum. Substrate 24 may alternatively be a polymeric material, such as poly(lactic acid), poly(glycolic acid), poly(dioxanone), poly(trimethylene carbonate), poly(ε-caprolactone), polyethylene, poly(etheretherketone), polyanhydrides, polyorthoesters, polyphosphazenes, or combinations thereof.

Drug-impregnated layer 26 may be a therapeutic substance on substrate 24 or a polymer with a therapeutic substance 30 dispersed throughout the polymer. Typically, a solution of the polymeric material and one or more therapeutic substances are mixed, often with a solvent, and the polymer mixture is applied to stent 10. Methods of applying the therapeutic substance or therapeutic substance and polymer mixture to strut 14 of stent 10 include, but are not limited to, immersion, spray-coating, sputtering, and gas-phase polymerization. Immersion, or dip-coating, entails submerging the entire stent 10, or an entire section, e.g., cylindrical ring 12, of stent 10, in the mixture. Stent 10 is then dried, for instance in a vacuum or oven, to evaporate the solvent, leaving the therapeutic substance or therapeutic substance and polymer coating on the stent. Similarly, spray-coating requires enveloping the entire stent, or an entire section of the stent, in a large cloud of the mixture, and then allowing the solvent to evaporate, to leave the coating. Sputtering typically involves placing a polymeric coating material target in an environment, and applying energy to the target such that polymeric material is emitted from the target. The polymer emitted deposits onto the device, forming a coating. Similarly, gas phase polymerization typically entails applying energy to a monomer in the gas phase within a system set up such that the polymer formed is attracted to a stent, thereby creating a coating around the stent. Drug-impregnated layer 26 may be in the range of about 0.5 to about 10 microns in thickness.

The polymer used for drug-impregnated layer 26 is preferably biodegradable. The term “biodegradable” as used in this application refers to materials that are capable of being completely degraded and/or eroded when exposed to bodily fluids such as blood and can be gradually resorbed, absorbed and/or eliminated by the body. The processes of breaking down and eventual absorption and elimination of the material can be caused by, for example, hydrolysis, metabolic processes, bulk or surface erosion, and the like. For coating applications, it is understood that after the process of degradation, erosion, absorption, and/or resorption has been completed, no material will remain on the device. In some embodiments, very negligible traces or residue may be left behind. Whenever the terms “degradable” or “biodegradable” are used in this application, they are intended to broadly include biologically erodable, bioabsorbable, and bioresorbable materials as well as other types of materials that are broken down and/or eliminated by the body. Examples of biodegradable materials include but are not limited to polycaprolactone (PCL), poly-D, L-lactic acid (DL-PLA), poly-L-lactic acid (L-PLA), poly(lactide-co-glycolide), poly(hydroxybutyrate), poly(hydroxybutyrate-co-valerate), polydioxanone, polyorthoester, polyanhydride, poly(glycolic acid), poly(glycolic acid-cotrimethylene carbonate), polyphosphoester, polyphosphoester urethane, poly (amino acids), cyanoacrylates, poly(trimethylene carbonate), poly(iminocarbonate), copoly(ether-esters), polyalkylene oxalates, polyphosphazenes, polyiminocarbonates, and aliphatic polycarbonates.

Therapeutic substance 30 may include, but is not limited to, antineoplastic, antimitotic, antiinflammatory, antiplatelet, anticoagulant, antifibrin, antithrombin, antiproliferative, antibiotic, antioxidant, and antiallergic substances as well as combinations thereof. Examples of such antineoplastics and/or antimitotics include paclitaxel (e.g., TAXOL® by Bristol-Myers Squibb Co., Stamford, Conn.), docetaxel (e.g., Taxotere® from Aventis S. A., Frankfurt, Germany), methotrexate, azathioprine, vincristine, vinblastine, fluorouracil, doxorubicin hydrochloride (e.g., Adriamycin® from Pharmacia & Upjohn, Peapack N.J.), and mitomycin (e.g., Mutamycin® from Bristol-Myers Squibb Co., Stamford, Conn.). Examples of such antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, and thrombin inhibitors such as Angiomax™(Biogen, Inc., Cambridge, Mass.). Examples of such cytostatic or antiproliferative agents include ABT-578 (a synthetic analog of rapamycin), angiopeptin, angiotensin converting enzyme inhibitors such as captopril (e.g., Capoten® and Capozide® from Bristol-Myers Squibb Co., Stamford, Conn.), cilazapril or lisinopril (e.g., Prinivil® and Prinzide® from Merck & Co., Inc., Whitehouse Station, N.J.), calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name Mevacor® from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), and nitric oxide. An example of an antiallergic agent is permirolast potassium. Other therapeutic substances or agents that may be used include nitric oxide, alpha-interferon, genetically engineered epithelial cells, and dexamethasone. In other examples, the therapeutic substance is a radioactive isotope for implantable device usage in radiotherapeutic procedures. Examples of radioactive isotopes include, but are not limited to, phosphorus (P³²), palladium (Pd¹⁰³), cesium (Cs¹³¹), Iridium (I¹⁹²) and iodine (I¹²⁵). While the preventative and treatment properties of the foregoing therapeutic substances or agents are well-known to those of ordinary skill in the art, the substances or agents are provided by way of example and are not meant to be limiting. Other therapeutic substances are equally applicable for use with the disclosed methods and compositions.

Barrier layer 28 acts to reduce the rate of delivery of therapeutic substance 30 to the internal target tissue area. Barrier layer 28 may be a biodegradable metal, biodegradable metal oxide or biodegradable metal alloy. In various embodiments, barrier layer 28 may be made from magnesium, iron, or an oxide or alloy of magnesium or iron. Several methods may be used to deposit barrier layer 28 on drug-impregnated layer 26, such as sputtering, plasma deposition, reactive sputtering, physical vapor deposition, chemical vapor deposition, or cathodic arc vacuum deposition, depending on the specific material used for barrier layer 28. Barrier layer 28 may have a thickness in the range from about 10 to about 100 nanometers. As shown in FIG. 2, drug-impregnated layer 26 and barrier layer 28 completely surround substrate 24.

FIG. 3 shows a cross-sectional view another embodiment of a strut 14′ of the stent 10 of FIG. 1 taken along line A-A. Strut 14′ is similar to strut 14 of FIG. 2 in that it includes a substrate 24, a drug-impregnated layer 26, and a barrier layer 28. However, drug impregnated layer 26 and barrier layer 28 are disposed on only one surface of strut 14′, preferably an outwardly facing surface 32 of substrate 24. As would be understood by one of ordinary skill in the art, drug-impregnated layer 26 and barrier layer 28 may cover other portions of substrate 24. For example, drug-impregnated layer 26 and barrier payer 28 may cover the outer and inner surfaces of substrate 24, but not the side surfaces, or may cover only the inner or outer surface, depending on the application.

FIG. 4 shows a cross-sectional view of another of a strut 14″ of the stent 10 of FIG. 1 taken along line A-A. Strut 14″ is similar to strut 14′ of FIG. 3 in that it includes a substrate 24, a drug-impregnated layer 26, and a barrier layer 28′. However, barrier layer 28′ is not a continuous surface. Instead, barrier layer 28′ comprises a number of discrete deposits above drug-impregnated layer 26, with the deposits separated by spaces 34. In the embodiment illustrated in FIG. 4, the rate of drug delivery from drug-impregnated layer 26 to the target area is reduced because the surface area for therapeutic substance 30 to diffuse from drug-impregnated layer 26 is reduced. The majority of drug therapeutic substance 30 will diffuse at spaces 34. Some of the therapeutic substance 30 will diffuse through barrier layer 28′, although at a slower rate than at spaces 34. Further, some of the therapeutic substance 30 located in drug-impregnated layer 26 below barrier layer 28′ will migrate to the area of spaces 34 to be delivered to the target tissue area. As would be understood by one of ordinary skill in the art, the embodiment of FIG. 4 may be modified such that drug-impregnated layer 26 and barrier layer 28′ cover all surfaces of strut 14″, similar to FIG. 2, or selected surfaces of strut 14″, as described above with respect to FIG. 3.

The embodiment illustrated in FIG. 4 may be achieved by performing deposition processes that deposit layers of material by way of nucleation, such as cathodic arc sputtering, reactive sputtering, thermal evaporation and electron beam (e-beam) evaporation. The embodiment illustrated in FIG. 4 may also be achieved by depositing a continuous film, and then creating holes in that film. For example, a magnesium film can be deposited with differing amounts of grain structure. An etching chemical (e.g., typically mixtures of mineral acids) may be used to preferentially etch between grains and remove some of the magnesium film. Alternatively, a continuous film could be deposited, and holes made in that continuous film by, for example, ion milling, a laser, or electron beam machining.

In an alternative method of tailoring the elution rate of the drug, similar to the embodiment of FIG. 4, the porosity of the barrier layer can be increased. In one method, wax or water soluble salt particles may be applied to the dried top surface of the drug-impregnated layer. The barrier layer is applied to the over the drug-impregnated layer. If salt particles are used, the salt can be washed away after the barrier layer is applied, thereby creating pores in the barrier layer. If wax particles are used, the wax particles may be left in place after application of the barrier layer. Upon deployment of the stent (expansion from it compressed configuration to its expanded configuration) the wax particles deform, thereby creating micro-cracks in the barrier layer. The micro-cracks alter the elution rate of the barrier layer.

A cross-sectional view of connecting links 22 of stent 10 may be similar to struts 14. 14′, 14″ or may be different. For example, a thickness of connecting links 22 may be different than strut 14 of cylindrical rings 12 to provide variable flexibility between the rings 12 and connecting links 22. A specific choice of thickness for struts 14 and links 22 depends on several factors, including, but not limited to, the anatomy and size of the target lumen. Further, struts 14, 14′, 14″ may be coated as described above and links 22 may be uncoated.

One of ordinary skill in the art will appreciate that, for all of the embodiments described herein, the thickness of barrier layer 28, 28′ may be varied, with a corresponding change in the drug release rate. Generally, the thicker the barrier, the greater the reduction in the drug release rate.

While various embodiments of the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. Furthermore, there is no intention to be bound by any expressed or implied theory presented in the preceding technical field, background, brief summary or the detailed description. All patents and publications discussed herein are incorporated by reference herein in their entirety.

1. An implantable medical device comprising:
a substrate;
a polymer coating disposed on said substrate, said polymer coating including a therapeutic substance; and
a barrier layer overlaying at least a portion of said polymer coating, wherein said barrier layer comprises a material selected from the group consisting of a biodegradable metal, a biodegradable metal oxide, and an alloy of a biodegradable metal.

2. The implantable medical device of claim 1, wherein said material of said barrier layer comprises magnesium.

3. The implantable medical device of claim 1, wherein said material of said barrier layer comprises a magnesium oxide.

4. The implantable medical device of claim 1, wherein said material of said barrier layer comprises a magnesium alloy.

5. The implantable medical device of claim 1, wherein said material of said barrier layer comprises iron.

6. The implantable medical device of claim 1, wherein said material of said barrier layer comprises an iron oxide.

7. The implantable medical device of claim 1, wherein said barrier layer is a continuous layer overlaying the polymer coating.

8. The implantable medical device of claim 1, wherein said barrier layer includes a plurality of discrete deposits overlaying the polymer coating.

9. The implantable medical device of claim 1, wherein said barrier layer has a thickness ranging from about 5 to about 100 nanometers.

10. The implantable medical device of claim 1, wherein said therapeutic substance is selected from the group consisting of antineoplastic, antimitotic, antiinflammatory, antiplatelet, anticoagulant, anti fibrin, antithrombin, antiproliferative, antibiotic, antioxidant, and antiallergic substances as well as combinations thereof.

11. The implantable medical device of claim 1, wherein said therapeutic substance is selected from the group consisting of alpha-interferon, genetically engineered epithelial cells, and dexamethasone.

12. The implantable medical device of claim 1, wherein said therapeutic substance is a radioactive isotope.

13. The implantable medical device of claim 1, wherein the medical device is a stent.

14. The implantable medical device of claim 1, wherein the medical device is a graft.

15. A method for making an implantable medical device comprising the steps of:
forming a first layer comprising a polymer and a therapeutic substance on the device; and
forming a barrier layer over at least a portion of said first layer, wherein said barrier layer comprises a material selected from the group consisting of a biodegradable metal, a biodegradable metal oxide, and an alloy of a biodegradable metal.

16. The method of claim 15, wherein said step of forming said barrier layer is a process selected from the group consisting of sputtering, plasma deposition, reactive sputtering, physical vapor deposition, chemical vapor deposition, and cathodic arc vacuum deposition.

17. The method of claim 16, wherein said barrier layer comprises a material selected from the group consisting of magnesium, a magnesium oxide, and a magnesium alloy.

18. The method of claim 15, wherein said barrier layer comprises a material selected from the group consisting of iron and an iron oxide.

19. A method of manufacturing a drug eluting stent comprising the steps of:
forming a reservoir layer containing a drug on a stent;
forming a barrier layer on said reservoir layer, wherein said barrier layer comprises a material selected from the group consisting of magnesium, a magnesium oxide, and a magnesium alloy.
